SEQUENCE LISTING (1 sequence)

SEQ ID NO: 1 - protein, 9 aa, Homo sapiens
TTLDSPLGK

(19) United States
(12) Patent Application Publication
(10) Pub. No.: US 2020/0271654 A1
(43) Pub. Date: Aug. 27, 2020

(54) Quantifying MGMT Protein For Optimal Cancer Therapy

(71) Applicant: Expression Pathology, Inc., Rockville, MD (US)

(72) Inventors: Todd HEMBROUGH, Gaithersburg, MD (US); Sarit SCHWARTZ, Rockville, MD (US); Fabiola CECCHI, Washington, DC (US)

(21) Appl. No.: 16/480,530

(22) PCT Filed: Feb. 6, 2018
(86) PCT No.: PCT/US18/17028

§ 371 Date: Jul. 24, 2019

Related U.S. Application Data
(60) Provisional application No. 62/455,479, filed on Feb. 6, 2017.

(51) Int. Cl. G01N 33/574; A61K 31/495; G01N 33/68

(52) U.S. Cl. CPC G01N 33/57446; A61K 31/495; G01N 2458/15; G01N 2333/91017; C12Y 304/21004; G01N 33/6848

(57) ABSTRACT

Methods are provided for identifying whether a tumor, and especially a colon tumor, will be responsive to treatment with the therapeutic agent temozolomide. A specific MGMT fragment peptide is precisely detected and quantitated by SRM-mass spectrometry directly in colon cancer cells collected from colon tumor tissue obtained from a cancer patient. Comparison to reference levels determines if the cancer patient will respond positively or negatively to treatment with the chemotherapeutic agent temozolomide.

### CROSS-REFERENCE TO RELATED APPLICATIONS

This application is a national stage application under 35 U.S.C. § 371 of PCT International Application No. PCT/US2018/017028, which has an International filing date of 6 Feb. 2018 and claims priority to U.S. Provisional Application No. 62/455,479 filed on 6 Feb. 2017. The contents of each application recited above are incorporated herein by reference in their entirety.

### FIELD

Improved methods for treating cancer patients are provided. Tumor tissue from a patient is assayed and the results of the assay are used to select an improved or optimal treatment regimen that is administered to the patient.

### BACKGROUND

Colorectal cancer (CRC) is the second leading cause of cancer-related death in the United States. Standard fluorouracil-based chemotherapy can prolong survival, but about half of CRC patients do not respond to these treatments, and those who do respond eventually become drug-resistant. The orally-administered chemotherapy drug temozolomide (TMZ) is a standard treatment for patients with melanoma and glioblastoma, but it has shown only modest efficacy in colorectal cancer. Temozolomide is an alkylating agent and is generally considered to be in the same class as chemotherapy agents such as cisplatin, carboplatin, oxaliplatin, cyclophosphamide, melphalan and chlorambucil.

The therapeutic benefit of temozolomide, as with other alkylating agents, depends on its ability to alkylate/methylate DNA, resulting in damage to the DNA with resultant triggering of cell death in tumor cells. Some tumor cells are able to repair this type of DNA damage, which diminishes the therapeutic efficacy of temozolomide. More specifically, cross-linking of double-stranded DNA by alkylating agents is inhibited by the cellular DNA-repair protein MGMT. In some tumors, epigenetic silencing of the MGMT gene prevents the synthesis of this enzyme and, as a consequence, such tumors are more sensitive to killing by temozolomide. Conversely, the presence of MGMT protein in glioblastoma tumor cells is associated with poor response to temozolomide and in these circumstances patients receive little benefit from temozolomide chemotherapy.

### SUMMARY

What is provided is a method of treating a patient suffering from colon cancer by (a) quantifying the level of a specified MGMT fragment peptide in a protein digest prepared from a tumor sample obtained from the patient and calculating the level of the MGMT fragment peptide in the sample by selected reaction monitoring using mass spectrometry, (b) comparing the level of the MGMT fragment peptide to a reference level, and (c) treating the patient with a therapeutic regimen containing an effective amount of the chemotherapeutic agent temozolomide when the level of the MGMT fragment peptide is lower than the reference level, or (d) treating the patient with a therapeutic regimen that does not contain an effective amount of the chemotherapeutic agent temozolomide when the level of the MGMT fragment peptide is above the reference level. The reference level of the MGMT fragment peptide may be 200 amol/μg, +/−200 amol/μg, +/−150 amol/μg, +/−100 amol/μg, +/−50 amol/μg, or +/−25 amol/μg, of biological sample protein analyzed. The specified MGMT peptide advantageously has the amino acid sequence as set forth as SEQ ID NO: 1. The tumor sample may be a cell, collection of cells, or a solid tissue and advantageously the tumor sample is formalin fixed solid tissue. The tissue may be paraffin embedded tissue.

In these methods, the protein digest may include a protease digest, such as a trypsin digest. The protein digest may be prepared by the Liquid Tissue protocol. The method mass spectrometry may include tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry, and the mode of mass spectrometry used may be, for example, Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).

Quantifying the specified MGMT fragment peptide may include determining the amount of the MGMT peptide in the sample by comparing to a spiked internal standard peptide of known amount, where both the native peptide in the biological sample and the internal standard peptide corresponds to the same amino acid sequence of the MGMT fragment peptide as shown in SEQ ID NO: 1. Advantageously the internal standard peptide is an isotopically labeled peptide, labeled with, for example, ¹⁸O, ¹⁷O, ¹⁵N, ¹³C, ²H and/or combinations thereof.

Detecting and quantitating the specified MGMT fragment peptide can be used to inform the treatment decision about which chemotherapy agent is used for treating a cancer patient and, optionally, can be combined with detecting and quantitating other peptides from other proteins in multiplex format so that the treatment decision about which agent used for treatment is based upon specific levels of the specified MGMT fragment peptide in combination with other peptides/proteins in the biological sample.

### BRIEF DESCRIPTION OF THE DRAWINGS

FIG. 1 shows overall survival curves where patients whose tumor tissue express levels of the MGMT protein <200 amol/μg of tumor cell protein have longer overall survival than patients whose tumor cells express above 200 amol/μg of tumor cell protein when treated with temozolomide. Results are shown as probability (0-100%) for overall survival in months after initiation of treatment. The Mantel-Cox log-rank and Gehan-Breslow-Wilcoxon tests were used for survival comparisons.

FIG. 2 shows progression-free survival curves where patients whose tumor cells obtained from patient tumor tissue express levels of the MGMT protein <200 amol/μg of tumor cell protein have longer progression-free survival than patients whose tumor cells express above 200 amol/μg of tumor cell protein when treated with temozolomide. Results are shown as probability (0-100%) for progression-free survival in months after initiation of treatment. The Mantel-Cox log-rank and Gehan-Breslow-Wilcoxon tests were used for survival comparisons.

### DETAILED DESCRIPTION

The presence and/or quantitative levels of MGMT protein expression in cells within tumor tissue is determined by quantitating a specified peptide derived from subsequences of the full-length MGMT protein (also referred to O⁶-alkylguanine DNA alkyltransferase, AGT, MGMT or AGAT). If expression of the MGMT protein is below a specified quantitative level, the patient is treated with a therapeutic regimen that includes the temozolomide therapeutic agent, and/or other drugs that function similarly to temozolomide. Alternatively, if the MGMT level is above the specified quantitative level, the patient is treated with a therapeutic regimen that does not include temozolomide, or other drugs that function similarly such as cisplatin, carboplatin, oxaliplatin, cyclophosphamide, melphalan and chlorambucil.

The MGMT peptide advantageously is detected using mass spectrometry-based Selected Reaction Monitoring (SRM), also referred to as Multiple Reaction Monitoring (MRM), referred to herein as an SRM/MRM assay. An SRM/MRM assay is used to detect the presence of, and quantitatively measure, the amount of a specified MGMT fragment peptide directly in cells procured from cancer patient tissue, such as, for example formalin fixed cancer tissue. Each molecule of unique MGMT fragment peptide is derived from one molecule of MGMT protein and therefore measurement of the amount of a specific unique MGMT peptide allows quantitation of the amount of intact MGMT protein in the tumor sample. Specific and optimized therapeutic agents and treatment strategies can be used to treat an individual cancer patient's disease based on the quantity of the MGMT protein is detected in their cancer cells.

More specifically, methods are provided for determining if a cancer patient will clinically respond in a favorable manner to the therapeutic cancer agent temozolomide. The quantitative level of the MGMT protein in a tumor sample or samples from the patient is measured. The sample is advantageously formalin-fixed and may be paraffin embedded.

The SRM/MRM assay advantageously measures a specified MGMT peptide fragment, and particular characteristics about the peptide, where the preferred peptide has the sequence TTLDSPLGK. Surprisingly it has been found that this peptide can be reliably detected and quantitated in digests prepared from formalin-fixed, paraffin embedded (“FFPE”) samples of tumor tissue. See U.S. patent application Ser. No. 13/993,045, the contents of which are hereby incorporated by reference in their entirety.

The SRM/MRM assay can be used to measure this peptide directly in complex protein lysate samples prepared from cells procured from patient tissue samples, such as formalin fixed cancer patient tissue. Methods of preparing protein samples from formalin-fixed tissue are described in U.S. Pat. No. 7,473,532, the contents of which are hereby incorporated by reference in their entirety. The methods described in U.S. Pat. No. 7,473,532 may conveniently be carried out using Liquid Tissue reagents and protocol available from Expression Pathology Inc. (Rockville, Md.).

The most widely and advantageously available form of tissue, and cancer tissue, from cancer patients is formalin fixed, paraffin embedded tissue. Formaldehyde/formalin fixation of surgically removed tissue is by far and away the most common method of preserving cancer tissue samples worldwide and is the accepted convention in standard pathology practice. Aqueous solutions of formaldehyde are referred to as formalin. “100%” formalin consists of a saturated solution of formaldehyde (about 40% by volume or 37% by mass) in water, with a small amount of stabilizer, usually methanol, to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature. Thus molecular analytical methods to analyze formalin fixed cancer tissue will be the most accepted and heavily utilized methods for analysis of cancer patient tissue.

Results from the SRM/MRM assay can be used to correlate accurate and precise quantitative levels of the MGMT protein within the specific cancer of the patient from whom the tissue was collected and preserved, including colon, skin, and brain cancer tissue. Advantageously, the levels of MGMT protein are measured in a tumor sample from colorectal cancer. This not only provides diagnostic information about the cancer, but also permits a physician or other medical professional to determine appropriate therapy for the patient. In this case, utilizing this assay can provide information about specific levels of MGMT protein expression in cancer tissue and whether or not the patient from whom the cancer tissue was obtained will respond in a favorable way to therapy with the anti-cancer therapeutic agent temozolomide, and/or potentially other similar drugs in the alkylating agent class designed to specifically damage DNA in tumor cells.

Treating cancer patients with temozolomide is one of the most common and effective strategies for preventing cancer from growing and thus prolonging the lives of cancer patients, especially brain and skin cancer patients. The MGMT protein is a protein that repairs damaged DNA; more specifically it repairs the type of damage inflicted by the anti-cancer therapeutic agent temozolomide, and other similar alkylating agents. Accordingly, if there is MGMT protein present in a tumor cell that is being treated with temozolomide, and/or other similar alkylating agents, the DNA will be constantly repaired and thereby provide chemotherapy resistance to the tumor cell and the tumor cell will likely not be killed. It therefore is useful for a clinician to know if MGMT protein is present in a patient's cancer cells because the effects of temozolomide, and/or other similar alkylating agents, will be negated and the cancer patient will not respond to temozolomide, and/or other similar alkylating agents.

Presently the most widely-used and applied methodology to determine protein presence in cancer patient tissue, especially FFPE tissue, is immunohistochemistry (IHC). IHC methodology utilizes an antibody to detect the protein of interest. The results of an IHC test are most often interpreted by a pathologist or histotechnologist. This interpretation is subjective and does not provide quantitative data that can be predictive of temozolomide sensitivity or resistance.

Research from other IHC assays, such as the Her2 IHC test, suggest the results obtained from such tests may be wrong. This is probably because different labs have different rules for classifying positive and negative IHC status. Each pathologist running the tests also may use different criteria to decide whether the results are positive or negative. In most cases, this happens when the test results are borderline, meaning that the results are neither strongly positive nor strongly negative. In other cases, tissue from one area of cancer tissue can test positive while tissue from a different area of the cancer tests negative. Inaccurate IHC test results may mean that patients diagnosed with cancer do not receive the best possible care. If all or part of a cancer is positive for a specific target oncoprotein but test results classify it as negative, physicians are unlikely to recommend the correct therapeutic treatment, even though the patient could potentially benefit from those agents. If a cancer is oncoprotein target negative but test results classify it as positive, physicians may recommend a specific therapeutic treatment, even though the patient is unlikely to get any benefits and is exposed to the agent's secondary risks.

In light of this there is great clinical value in the ability to correctly evaluate accurate quantitative levels of the MGMT protein in tumors, especially brain, skin, and colon tumors, so that the patient will have the greatest chance of receiving the correct chemotherapy that may or may not include treatment with temozolomide, and/or other similar alkylating agents.

Detecting and determining quantitative levels of the specified MGMT fragment peptide is performed in a mass spectrometer by the SRM/MRM methodology, whereby the SRM/MRM signature chromatographic peak area of the peptide is determined within a complex peptide mixture present in a Liquid Tissue lysate (see U.S. Pat. No. 7,473,532, as described above). Quantitative levels of the MGMT protein is then determined by the SRM/MRM methodology whereby the SRM/MRM signature chromatographic peak area of an individual specified peptide from the MGMT protein in one biological sample is compared to the SRM/MRM signature chromatographic peak area of a known amount of a “spiked” internal standard for the individual MGMT fragment peptide.

In one embodiment, the internal standard is a synthetic version of the same exact MGMT fragment peptide where the synthetic peptide contains one or more amino acid residues labeled with one or more heavy isotopes. Such isotope labeled internal standards are synthesized so that mass spectrometry analysis generates a predictable and consistent SRM/MRM signature chromatographic peak that is different and distinct from the native MGMT fragment peptide chromatographic signature peaks and which can be used as a comparator peak. When the internal standard is spiked in known amounts into a protein or peptide preparation from a biological sample and analyzed by mass spectrometry, the SRM/MRM signature chromatographic peak area of the native peptide is compared to the SRM/MRM signature chromatographic peak area of the internal standard peptide, and this numerical comparison indicates either the absolute molarity and/or absolute weight of the native peptide present in the original protein preparation from the biological sample. Quantitative data for fragment peptides are displayed according to the amount of protein analyzed per sample.

Additional information beyond the peptide sequence is needed to develop the SRM/MRM assay for the MGMT fragment peptide. That additional information is used to direct the mass spectrometer, (e.g., a triple quadrupole mass spectrometer) to perform the correct and focused analysis of the specified MGMT fragment peptide. An SRM/MRM assay advantageously is performed on a triple quadrupole mass spectrometer, which is the most suitable instrument for analyzing a single isolated target peptide within a very complex protein lysate that may consist of hundreds of thousands to millions of individual peptides from all the proteins contained within a cell. Although the most advantageous instrument platform for SRM/MRM assay is typically considered to be a triple quadrupole instrument, SRM/MRM assays can be developed and performed on any type of mass spectrometer, including a MALDI, ion trap, ion trap/quadrupole hybrid, or triple quadrupole. The additional information about target peptides in general, and in particular about the specified MGMT (SEQ ID NO: 1) fragment peptide, may include one or more of the mono isotopic mass of each peptide, its precursor charge state, the precursor m/z value, the m/z transition ions, and the ion type of each transition ion. The peptide sequence of the specified MGMT fragment peptide and the necessary additional information as described for this specified MGMT fragment peptide is shown in Table 1.

TABLE 1

| | | Mono | Precursor | | | |
|---|---|---|---|---|---|---|
| | Peptide | Isotopic | Charge | Precursor | Transition | Ion |
| SEQ ID | sequence | Mass | State | m/z | m/z | Type |
| SEQ ID NO: 1 | TTLDSPLGK | 930.5021 | 2 | 466.258 | 414.27 | Y4 |
| | | | 2 | 466.258 | 616.329 | Y6 |
| | | | 2 | 466.258 | 729.413 | Y7 |

To determine an appropriate reference level for MGMT quantitation, tumor samples are obtained from a cohort of patients suffering from cancer, in this case colon cancer. The colon tumor samples are formalin-fixed using standard methods and the level of MGMT in the samples is measured using the methods as described above. The tissue samples may also be examined using IHC and FISH using methods that are well known in the art. The patients in the cohort are treated with the temozolomide therapeutic agent and the response of the patients is measured using methods that are well known in the art, for example by recording the progression-free survival and overall survival of the patients at time intervals after treatment. A suitable reference level can be determined using statistical methods that are well known in the art, for example by determining the lowest p value of a log rank test.

Once a reference level has been determined it can be used to identify those patients whose MGMT expression levels indicate that they may likely benefit from a temozolomide therapeutic regimen. The skilled artisan will recognize that temozolomide is also used as part of a treatment regimen that utilizes additional drugs or combinations of drugs, such as in combination with an inhibitor of the EGFR protein. Therapeutic regimens for treating colon cancer are known in the art. For example, treatment regimens that do not include an effective amount of temozolomide include: FOLFOX (leucovorin, 5-FU, and oxaliplatin); FOLFIRI (leucovorin, 5-FU, and irinotecan); CapeOX: (capecitabine and oxaliplatin), or FOLFOXIRI: (leucovorin, 5-FU, oxaliplatin, and irinotecan). Any of these regimens can be combined with a targeted drug, such as a drug that targets VEGF (such as bevacizumab, ziv-aflibercept, or ramucirumab), or a drug that targets EGFR (such as cetuximab or panitumumab). Other regimens that optionally may be used with a targeted drug include 5-FU and leucovorin, capecitabine, and irinotecan. Additional regimens that may be used include cetuximab alone, panitumumab alone, regorafenib alone, or a combination of trifluridine and tipiracil.

Levels of the MGMT protein in patient tumor samples are typically expressed in amol/μg, although other units can be used. The skilled artisan will recognize that a reference level can be expressed as a range around a central value, for example, +/−250, 150, 100, 50 or 25 amol/μg. In the specific example described in detail below a suitable reference level for the MGMT protein was found to be 200 amol/μg which is the lower limit of quantitation. However, the skilled artisan will recognize that levels higher or lower than these reference levels can be selected based on clinical results and experience.

Because both nucleic acids and protein can be analyzed from the same Liquid Tissue biomolecular preparation it is possible to generate additional information about disease diagnosis and drug treatment decisions from the nucleic acids in same sample upon which proteins were analyzed. For example, if the MGMT protein is expressed by certain cells at increased/decreased levels, when assayed by SRM the data can provide information about the state of the cells and their potential for uncontrolled growth, choice of optimal therapy, and potential drug resistance. At the same time, information about the status of genes and/or the nucleic acids and proteins they encode (e.g., mRNA molecules and their expression levels or splice variations) can be obtained from nucleic acids present in the same Liquid Tissue biomolecular preparation.

Nucleic acids can be assessed simultaneously to the SRM analysis of proteins, including the MGMT protein. In one embodiment, information about the MGMT protein and/or one, two, three, four or more additional proteins may be assessed by examining the nucleic acids encoding those proteins. Those nucleic acids can be examined, for example, by one or more, two or more, or three or more of: sequencing methods, polymerase chain reaction methods, restriction fragment polymorphism analysis, identification of deletions, insertions, and/or determinations of the presence of mutations, including but not limited to, single base pair polymorphisms, transitions, transversions, or combinations thereof.

### Example: Determination of a Predictive Value of MGMT Protein Expression for Temozolomide Sensitivity/Resistance in a Population of Colon Cancer Patients

### Patients

24 patients from Fondazione IRCCS Istituto Nazionale dei TumoriVia Venezian, Milan, Italy, were identified with advanced colorectal cancer (CRC). Tumors were surgically removed prior to treatment and archived as formalin-fixed, paraffin-embedded (FFPE) tissue and all were histologically colorectal cancer. All 24 patients were subsequently treated with temozolomide.

### Methods

Archived formalin-fixed, paraffin-embedded tissue sections were obtained from patients with advanced CRC treated with temozolomide (n=24). A board-certified pathologist marked the tumor areas. Based on the markups, tumor cells were laser microdissected and solubilized to tryptic peptides using the Liquid Tissue process. In each liquefied sample, multiple protein biomarkers including MGMT were quantified by selected reaction monitoring (SRM) mass spectrometry (MS). A mixture of stable isotope-labeled heavy peptides including the MGMT fragment peptide TTLDSPLGK was added prior to MS analysis. Each sample was analyzed in triplicate. Statistical analysis of the results indicated that a MGMT cutoff of 200 amol/μg protein analyzed, based on the limit of quantitation from a concentration curve. The Mantel-Cox log-rank and Gehan-Breslow-Wilcoxon tests were used for survival comparisons.

### Results

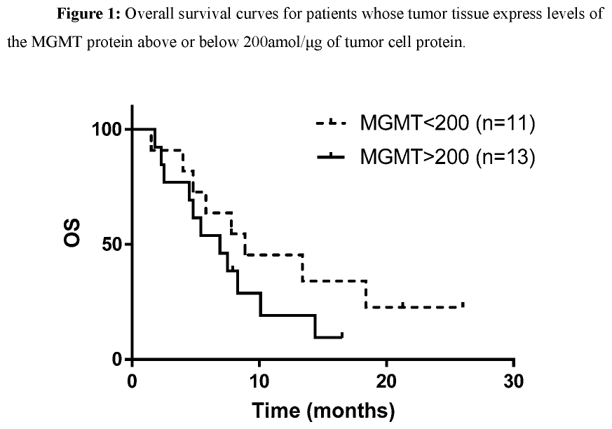

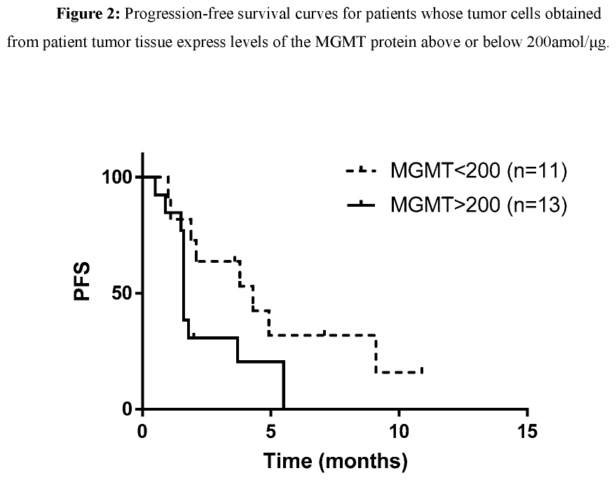

The MGMT fragment peptide was detected in 13 of 24 (54.2%) colorectal tumor samples (range: 229.3-784.8 amol/μg). CRC patients with MGMT protein levels below a cutoff of 200 amol/μg (n=11) had approximately 2.5 times the median progression-free survival (PFS) as patients with MGMT levels above the cutoff (4.3 vs. 1.6 months, p=0.0611), while a slight difference was observed in overall survival. Results indicated a correlation of increased MGMT protein expression with a negative therapeutic outcome by treatment of the cancer patient with temozolomide as evidenced by decreased progression-free survival and overall survival after initial diagnosis and initiation of treatment regime with temozolomide (FIGS. 1-2). Likewise, lower levels of MGMT correlated with a positive therapeutic outcome as evidenced by increased progression-free survival and overall survival from treatment with temozolomide (FIGS. 1-2). Quantitative levels of other analyzed proteins did not correlate with a negative/positive outcome to treatment with temozolomide.

1. A method of treating a patient suffering from colon cancer comprising:
(a) quantifying a level of a MGMT fragment peptide in a protein digest prepared from a tumor sample obtained from the patient and calculating a level of the MGMT fragment peptide in said tumor sample by selected reaction monitoring using mass spectrometry;
(b) comparing the level of said MGMT fragment peptide to a reference level, and
(c) treating the patient with a therapeutic regimen comprising an effective amount of temozolomide when the level of the MGMT fragment peptide is lower than said reference level, or
(d) treating the patient with a therapeutic regimen that does not comprise an effective amount of temozolomide when the level of the MGMT fragment peptide is above said reference level.

2. The method of claim 1, wherein said reference level of the MGMT fragment peptide is 200 amol/μg, +/−200 amol/μg, of sample protein analyzed.

3. The method of claim 1, wherein said reference level is 200 amol/μg, +/−150 amol/μg, of sample protein analyzed.

4. The method of claim 1, wherein said reference level is 200 amol/μg, +/−100 amol/μg, of sample protein analyzed.

5. The method of claim 1, wherein said reference level is 200 amol/μg, +/−50 amol/μg, of sample protein analyzed.

6. The method of claim 1, wherein said reference level is 200 amol/μg, +/−25 amol/μg, of sample protein analyzed.

7. (canceled)

8. The method of claim 1, wherein said protein digest comprises a protease digest.

9. The method of claim 8, wherein said protein digest comprises a trypsin digest.

10. The method of claim 1, wherein mass spectrometry comprises tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry.

11. The method of claim 10, wherein a mode of mass spectrometry used is Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).

12. The method of claim 1, wherein the MGMT peptide has the amino acid sequence as set forth as SEQ ID NO: 1.

13. The method of claim 1, wherein the tumor sample is a cell, collection of cells, or a solid tissue.

14. The method of claim 13, wherein the tumor sample is formalin fixed solid tissue.

15. The method of claim 14, wherein the tissue is paraffin embedded tissue.

16. The method of claim 1, wherein quantifying the MGMT fragment peptide comprises determining an amount of the MGMT fragment peptide in said tumor sample by comparing to a spiked internal standard peptide of known amount, wherein both the MGMT fragment peptide in said tumor sample and the internal standard peptide corresponds to the same amino acid sequence of the MGMT fragment peptide as shown in SEQ ID NO: 1.

17. The method of claim 16, wherein the internal standard peptide is an isotopically labeled peptide.

18. The method of claim 17, wherein the isotopically labeled internal standard peptide comprises one or more heavy stable isotopes selected from ¹⁸O, ¹⁷O, ¹⁵N, ¹³C, ²H or a combinations thereof.

19. The method of claim 16, wherein detecting and quantifying the MGMT fragment peptide can be used to inform a treatment decision about which chemotherapy agent is used for treating the cancer patient.

20. The method of claim 19, wherein detecting and quantitating the MGMT fragment peptide can be combined with detecting and quantitating other peptides from other proteins in a multiplex format so that the treatment decision about which agent used for treatment is based upon levels of the MGMT fragment peptide in combination with other peptides/proteins in the tumor sample.
